(19) United States
(12) Patent Application Publication
(10) Pub. No.: US 2012/0203268 A1
(43) Pub. Date: Aug. 9, 2012

(54) Wrinkle remover

(76) Inventor: Lin Chien, Vienna, VA (US)

(21) Appl. No.: 13/022,016

(22) Filed: Feb. 7, 2011

(51) Int. Cl. A61F 5/08

(52) U.S. Cl. 606/204.35

(57) ABSTRACT

A wrinkle removing device consists of a plastic strip or patch with glue on one side, a button or hook and a single band of elastic material. The patch has a glue side and an attachment side. The glue side is for securing to a person's skin under the hairline. A button or hook is attached to the glue side and within the confines of the plastic patch. The hook provides an arced member or open capture slot immediately adjacent to the attachment side of the plastic patch. The single band of elastic material is removably attachable within the open capture slot of said button or hook and is stretched to fit over or around a person's head, thus pulling on the plastic patches to stretch the skin.

### TECHNICAL FIELD

In the field of head coverings, a device is used to lift facial or neck skin to remove wrinkles.

### BACKGROUND ART

Skin lifting devices that remove wrinkles from the face or neck of a person to add a youthful look to a person have been disclosed. These, however, are complicated devices typically with multiple loops and stretchable links, tabs that add weight and bulk to the device, and clasps that bind the stretchable material together but can dig into a person's head and be uncomfortable during use.

### SUMMARY OF INVENTION

A wrinkle removing device consists of two or more plastic strips or patches. Each patch has a glue side for securing to a person's skin under the hairline and a glue side. A button or hook is attached to the glue side and within the confines of each plastic patch. The button or hook provides an arced member defining an open capture slot immediately adjacent to the attachment side of each plastic patch. A single loop or band of elastic material is removably attachable within the open capture slot of each said button or hook and is stretched to fit over or around a person's head, thus pulling on the plastic patches to stretch the skin.

### Advantageous Effects of Invention

A simple, lightweight and uncomplicated and comfortable device is taught that makes wrinkle removing easy to accomplish. Sagging skin on the neck can easily become taught using the device and a person can appear years younger. The device can remain attached to the skin and hidden from observers for extended periods of time without discomfort on the part of the user.

### BRIEF DESCRIPTION OF DRAWINGS

The drawings illustrate preferred embodiments of the invention and the reference numbers in the drawings are used consistently throughout. New reference numbers in FIG. 2 are given the 200 series numbers. Similarly, new reference numbers in each succeeding drawing are given a corresponding series number beginning with the figure number.

FIG. 1 is a front view of the device shown installed in one position on the head of person.

FIG. 2 is a top view of a band of elastic material comprising a component of the device.

FIG. 3 is a top and side view of a plastic patch and attached button comprising two components of the device.

FIG. 4 is a top and side view of a plastic patch and attached hook comprising two components of an alternative device.

FIG. 5 is a side elevation view of a plastic patch and attached button comprising two components of the device.

FIG. 6 is a side elevation view of a plastic patch and attached hook comprising two components of an alternative device.

### DESCRIPTION OF EMBODIMENTS

In the following description, reference is made to the accompanying drawings, which form a part hereof and which illustrate several embodiments of the present invention. The drawings and the preferred embodiments of the invention are presented with the understanding that the present invention is susceptible of embodiments in many different forms and, therefore, other embodiments may be utilized and structural, and operational changes may be made, without departing from the scope of the present invention.

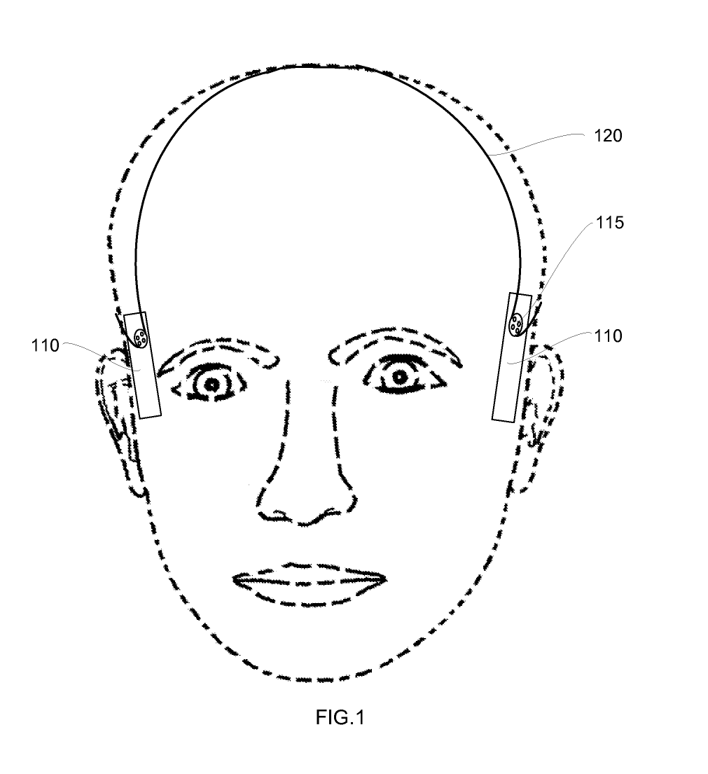

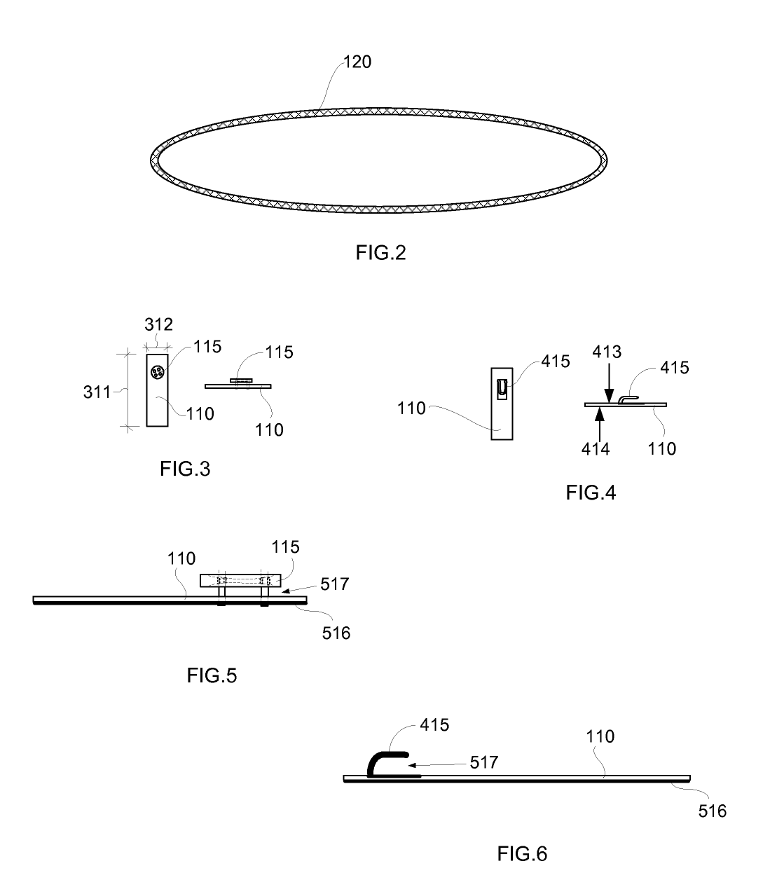

FIG. 1 shows a front view of a preferred embodiment of the wrinkle removing device installed in an illustrative position on the figurative head of person. There are four components of the wrinkle remover: a plurality of plastic patches (110); glue (516) on an attachment side of each patch as shown in FIG. 5.; a button (115) or hook (415) as shown in FIG. 1 and FIG. 4, respectively; and a band (120) of elastic material. While a preferred embodiment uses two patches, one on each side of a person's head or neck, an alternative embodiment includes a single patch with a button (115) or hook (415) thereon, for example for use at the top of a person's forehead.

Whether for embodiments with one or a plurality of plastic patches (110), each plastic patch is preferably a thin, transparent, durable and rubber-like material, commonly found in bandages, that is well known and commonly available. A skin-colored patch may also be used to match the varied colors of human skin.

To simplify the discussion herein, the explanation is based on using a plurality of patches because embodiments using a plurality of patches are considered the best use of the invention. However, it should be recognized that a single patch may be used and is within the scope of the invention.

There are preferably two plastic patches in the plurality of plastic patches (110), which are attachable to a user's skin on each side of the user's head. More than two patches may be used if the user desires to stretch the skin on the face and neck at the same time. Each plastic patch in the plurality of plastic patches (110) comprises a length (311); a width (312); an attachment side (413); and a glue side (414). The actual size of each such patch may vary for comfort in use on the head of a user and to maximize the ability to conceal the patch under a user's hair.

The preferred wrinkle remover includes glue affixed to the glue side of each plastic patch within the plurality of plastic patches (110). The glue is also well known on bandages for adhering to a person's skin.

The hook (415) secured to the attachment side (413) and within the length (311) and width (312) of each plastic patch in the plurality of plastic patches (110). The hook (415) is preferably a simple wire type hook commonly found on women's clothes. The hook (415) is preferably sewn on each such patch, but other means of attachment, such as gluing, may be used. The hook (415) defines an open capture slot (517) immediately adjacent to the attachment side (413) of each plastic patch in the plurality of plastic patches (110).

The band (120) of elastic material is removably attachable within the open capture slot (517) of each hook (415). It is a single strand of elastic material much like a rubber band. The wrinkle remover uses only one loop as the band (120) of elastic material, not more than one. The band (120) of elastic material is a single continuous loop of stretchable rope-like material. Such a stretchable loop is commonly available in all sizes, for example, in securing a pony tail in a person's hair.

In an alternative embodiment of the wrinkle remover, the hook (415) is replaced with the button (115), which is preferably sewn on to each plastic patch in the plurality of plastic patches (110). Since the preferred plastic patch is a durable, usually stretchable plastic or durable rubber-like material, sewing as a means of attachment is possible. As with any button attached to a shirt or other garment, it must be sewn on so as to leave a space, or open capture slot (517), underneath the button to accommodate engagement with the band (120) of elastic material. Thus, the button and the hook are alternative means of attachment within the open capture slot (517) for the band (120) of elastic material. Any other means of attachment that enables the band (120) of elastic material to be easily attached and removed from each such plastic patch in the plurality of plastic patches (110) may be used.

For both embodiments, the band (120) of elastic material is large enough to fit around a person's head so that it can both engage the hook (415) or button (115) and remain in place encircling the person's head just by virtue of spanning the person's head within the person's hair lines. The button (115) or hook (415) should be as small as possible to minimize its weight and maximize the potential to hide it under a person's hair line. Thus, these means of attachment for the band (120) of elastic material should be small enough to fit within the length (311) and width (312) of each plastic patch in the plurality of plastic patches (110), yet also easily and securely engage the band (120) of elastic material in the open capture slot (517) provided by the hook (415) or button (115).

In a preferred method of using the wrinkle remover, a person secures two patches with a button (115) or hook (415) thereon to opposite sides of their cheek or neck, for example as shown in FIG. 1. The person attaches the band (120) using each open capture slot (517) provided by each hook (415) or button (115). The person stretches the band around their head, typically under the hair line, in such a manner as to secure the band in place around the person's head so that the band (120) stays in place and pulls on the two patches. The band (120), thus, pulls the person's skin taut and such action removes sags and wrinkles. It is also possible to use a single patch with a button (115) or hook (415) thereon, for example, at the top of the forehead and stretch the band around the head to pull on the forehead skin.

### Industrial Applicability

The invention has application to the beauty products industry.

What is claimed is:

1. A wrinkle removing device consisting of:
a plastic patch comprising a length; a width; an attachment side; and a glue side;
glue affixed to the glue side of the plastic patch;
a hook or button attached to the attachment side and within the length and width of the plastic patch, wherein said hook or button defines an open capture slot immediately adjacent to the attachment side of each said plastic patch; and
a band of elastic material removably attachable within the open capture slot of said hook or button.

2. A wrinkle removing device consisting of:
a plurality of plastic patches wherein each plastic patch in the plurality of plastic patches comprises a length; a width; an attachment side; and a glue side;
glue affixed to the glue side of each plastic patch within the plurality of plastic patches;
a hook or button attached to the attachment side and within the length and width of each said plastic patch, wherein said hook or button defines an open capture slot immediately adjacent to the attachment side of each said plastic patch; and
a band of elastic material removably attachable within the open capture slot of each said hook or button.
